(19) United States
(12) Patent Application Publication
(10) Pub. No.: US 2026/0026738 A1
(43) Pub. Date: Jan. 29, 2026

(54) METHOD FOR MONITORING FACIAL SKIN CONDITION

(71) Applicants: NATIONAL TAIPEI UNIVERSITY OF TECHNOLOGY, TAIPEI CITY (TW); TAIPEI MEDICAL UNIVERSITY, TAIPEI CITY (TW)

(72) Inventors: CHENG-CHUN CHANG, TAIPEI CITY (TW); SYU-JYUN PENG, TAIPEI CITY (TW); CHIA-LUN CHOU, TAIPEI CITY (TW)

(21) Appl. No.: 19/035,263

(22) Filed: Jan. 23, 2025

(30) Foreign Application Priority Data
Jul. 24, 2024 (TW) 113127639

(51) Int. Cl. A61B 5/00; G06T 7/00; G16H 15/00; G16H 20/30; G16H 30/40; G16H 40/63

(52) U.S. Cl. CPC A61B 5/445; A61B 5/0064; A61B 5/7405; A61B 5/742; G06T 7/0012; G16H 15/00; G16H 20/30; G16H 30/40; G16H 40/63; G06T 2207/10012; G06T 2207/20221; G06T 2207/30168; G06T 2207/30201

(57) ABSTRACT

In a monitoring method of facial skin condition, facial pictures are taken at multiple orientations of the face with a portable information device to produce face images. A quality check of the face images is conducted with an image quality check model. An instruction to take a new facial picture for replacing a corresponding face image failing in the quality check is automatically given. Repeat the steps of taking facial pictures and conducting the quality check until a required number of qualified face images are collected, and then automatically combine the collected qualified face images into a planar panoramic face image and automatically conducting a recognition process, a counting process and a classifying process of skin lesions of the patient based on the planar panoramic face image to generate a data file. An AI model is used to interpret and analyze contents of the data file to produce an inspection report of the facial skin condition.

### FIELD OF THE INVENTION

The present invention relates to a method for monitoring a facial skin condition, and more particularly to a method for automatically monitoring a facial skin condition of a patient.

### BACKGROUND OF THE INVENTION

Acne, also known as “pimples”, is a very common disease in dermatology outpatient clinics, and almost all people have ever suffered from the skin condition problem during the growth process. Secretion from sebaceous glands increases rapidly due to effects of hormones, and the most common period of incidence is between 12 and 25 years old. Acne is often caused by diet, lifestyle, emotional stress, endocrine or climatic changes, use of oral medications or inappropriate maintenance products, and recurs on the face, causing great distress to patients.

However, less than 40% of patients actually seek medical treatment for Acne, and less than half of those who do well follow doctor's instructions for medication. One of the possible reasons is that Acne patients lack of awareness that their symptoms have been serious to a degree requiring special attention to daily maintenance or medication. Another possible reason is that Acne patients or their caregivers lack of knowledge about clinical classification of lesions and their severity. Lack of picture records for reference and comparison during the disease course might also result in missing the timing for treatment of serious inflammation. Inflammatory skin lesions not only cause physical discomfort and distress of the patients, but also cause psychological and social problems of the patients once permanent scars are rendered. According to research literature, Acne patients have a relatively high risk of depression, anxiety, or suicidal ideation, and the quality of life would be adversely affected.

In the current clinical diagnosis process, dermatologists manually identify and categorize acne lesions, and then assess the severity of acne lesions by counting the number of lesions on the entire face. However, it is actually hard to obtain an accurate count. Since the counting process is time- and labor-consuming, it is difficult to accurately count a large number of lesions, and it is likely to overlook the ones in any blind area on the face, or repetitively accumulate the number particularly at junctions.

On the other hand, accurate counting conducted with professional skin medical equipment would be costly and can only be operated by a specialist. In addition, it would take a long time to wait for both clinical inspection and diagnosis report.

### SUMMARY OF THE INVENTION

Therefore, a method which can be readily, routinely and conveniently conducted to monitor a facial skin condition is provided according to the present invention.

In an aspect of the present invention, a method for monitoring a facial skin condition of a patient includes: taking facial pictures of the patient at multiple orientations of the patient's face with a portable information device to produce a plurality of face images; conducting a quality check of the plurality of face images with an image quality check model; automatically giving an instruction to take a new facial picture for replacing a corresponding face image failing in the quality check; repeating the steps of taking facial pictures and conducting the quality check until a required number of qualified face images are collected by the portable information device; automatically combining the collected qualified face images into a planar panoramic face image; automatically conducting a recognition process, a counting process and a classifying process of skin lesions of the patient based on the planar panoramic face image to generate a data file; and interpreting and analyzing contents of the data file with an artificial intelligence model to produce an inspection report of the facial skin condition.

In another aspect of the present invention, a method for monitoring a facial skin condition of a patient includes: creating a panoramic face image of the patient with a portable information device; conducting a quality check of the panoramic face image with an image quality check model; automatically giving an instruction to create a new panoramic face image for replacement if the quality check determines a failure result; repeating the steps of creating the panoramic face image and conducting the quality check until the panoramic face image passes the quality check; automatically conducting a recognition process, a counting process and a classifying process of skin lesions of the patient based on the panoramic face image passing the quality check to generate a data file; and interpreting and analyzing contents of the data file with an artificial intelligence model to produce an inspection report of the facial skin condition.

In further aspect of the present invention, a method for monitoring a facial skin condition of a patient includes: taking three-dimensional pictures from a plurality of orientations of the patient's face with a portable information device, thereby producing a first stereo model file of face image, wherein the stereo model file of face image contains a plurality of pixel points, each corresponding to a three-dimensional coordinate; conducting a quality check of the panoramic face image with an image quality check model; repeating the steps of taking the three-dimensional pictures and conducting the quality check until a second stereo model file of face image, which passes the quality check, is obtained; automatically conducting a recognition process, a counting process and a classifying process of skin lesions of the patient based on the second stereo model file of face image to generate a data file; and interpreting and analyzing contents of the data file with an artificial intelligence model to produce an inspection report of the facial skin condition.

### BRIEF DESCRIPTION OF THE DRAWINGS

The above contents of the present invention will become more readily apparent to those ordinarily skilled in the art after reviewing the following detailed description and accompanying drawings, in which:

FIG. 1 is a systematic block diagram schematically illustrating a working environment where a method of monitoring a facial skin condition according to the present invention can be applied; and

FIG. 2 is a flowchart schematically illustrating a method of monitoring a facial skin condition according to an embodiment of the present invention.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only; it is not intended to be exhaustive or to be limited to the precise form disclosed.

In the following, some specific embodiments are selected to illustrate the implementation of the present invention. Those skilled in the art can clearly understand the technical features and effects of the present invention from the contents disclosed in this specification. However, it should be noted that the configurations, proportions, sizes, etc., shown in the accompanying drawings are only for illustrating the disclosure in the specification to an extent that those skilled in the art can understand, but they are not intended to be confined to those exactly shown in the drawings. Any modification of shapes, change of proportions or adjustment of sizes shall be included in the scope of the invention disclosed in this specification. Furthermore, in this specification, the expressions such as “top”, “bottom”, “left”, “right”, “center”, etc. are used as spatial reference only, but not indicate positions. Likewise, the expression “a” or “an” is used only for specifying a certain type, but not limited to the number “one”.

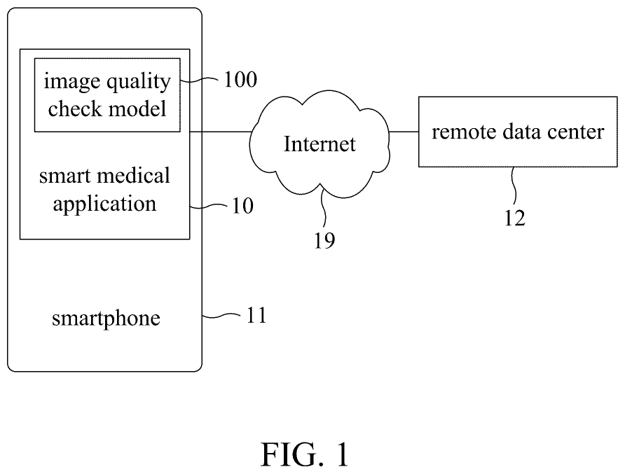

In order to solve the above-described drawbacks, a smart medical application related to skin care that can be conveniently used at the patient's end is developed according to the present invention. Refer to FIG. 1, which is a schematic block diagram illustrating a working environment in which the present invention can be used. The skin care-related smart medical application 10, for example, can be installed in a user's device, e.g., a smartphone 11, or any other suitable portable information device such as tablet, laptop, etc., as long as a camera module for picking up images of a user is included. The data generated by the skin care-related smart medical application 10 in response to the captured images can be transmitted from the smartphone 11 to a remote data center 12 via the Internet 19 for subsequent processing. In an embodiment, the smart medical application 10 is built therein an image quality check model 100, which can be used to perform an image quality check on the captured images. The image quality check may include a focus check, a hand-held shake check, and an exposure value check, but not limited thereto. By way of the focus check, hand-held shake check and exposure value check, factors possibly resulting in image defects, for example but not limit to, blurring, overexposure, underexposure, can be analyzed. Accordingly, the user can be instructed by the smart medical application 10 to modify the image-capturing operations, in order that pictures with satisfactory image quality can be obtained consequently. The image quality check model 100 can also be implemented with an image processing artificial intelligence (AI) model, which can be built directly into the smartphone 11 at the edge, and integrated with the smart medical application 10. Alternatively, the smart medical application 10 provided according to the present invention can be integrated into a portrait selfie application built in the smartphone 11 to execute an unnoticeable monitoring operation in the background while the user is taking a selfie with the portrait selfie application. In an embodiment, the smart medical application 10 also conducts a collecting function automatically during taking selfies to collect only images of the user's face, which are complete and pass the quality check. Subsequently, by combining these collected face images, a planar panoramic face image of the user can be created.

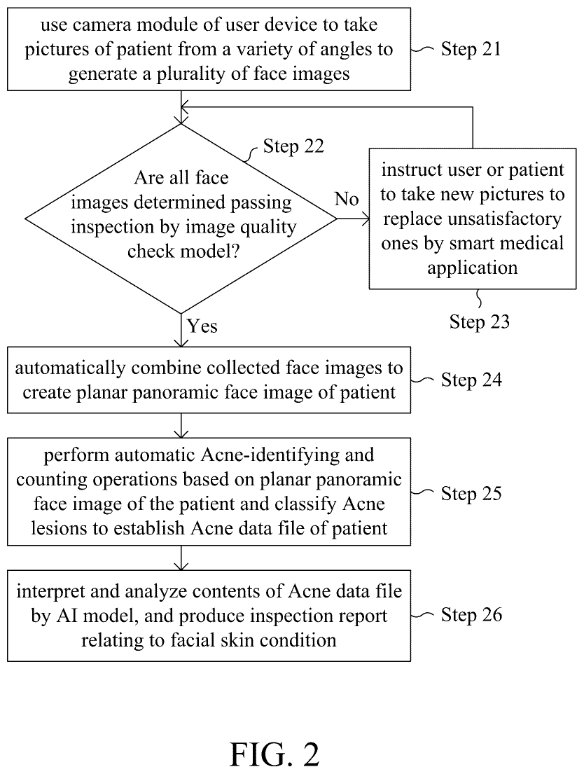

A flowchart show in FIG. 2 schematically illustrates a method of monitoring a facial skin condition according to an embodiment of the present invention. The method can be performed by a user or a patient with a user device, and mainly includes the following steps. First, the camera module of the user device is used to take pictures of the patient from a variety of angles to generate a plurality of face images (Step 21). The plurality of face images are inspected by the image quality check model 100 to determine if there is any face image failing in the inspection (Step 22). In Step 23, the smart medical application 10 instructs the user or the patient to take new pictures to replace the unsatisfactory ones. The above steps are repeated until a sufficient number of required and qualified facial images are obtained, and then proceed to Step 24. In step 24, the collected face images are automatically combined to create a planar panoramic face image of the patient. As a result, a high-quality image of the entire face of the patient can be obtained. Based on the planar panoramic face image of the patient, automatic Acne-identifying and counting operations are performed. The Acne lesions may further be classified according to their characteristics. Accordingly, an Acne data file of the patient is established (Step 25). Subsequently, the contents of the Acne data file are interpreted and analyzed by an artificial intelligence (AI) model, and accordingly, an inspection report relating to the facial skin condition is produced (Step 26). The inspection report may include, for example, a medical advice about the need to see a doctor, and a daily care advice about Acne care and skin maintenance. In other words, the patient only needs to take pictures with the smartphone 11, and then the AI model built in the smartphone 11 (and alternatively, a remote AI model) will automatically determine the severity of the acne lesions in real time. In an embodiment, scars resulting from Acne may also be found and located to be particularly treated. Therefore, according to the present invention, daily records of the patient's acne lesions can be established, and an adequate medical advice can be given based on the records, where the severity of acne lesions is indicated. It is important to seek medical treatment in time to prevent Acne lesions from turning into scars. In addition, comparison of the skin condition before and after treatment can also be conducted based on the records. According to an embodiment of the present invention, a reminder function can be set up during medical treatment to remind the patient of taking routine actions, e.g., taking medications or doing blood tests in due course. Furthermore, instructions and/or reminders about daily care may also be provided according to an embodiment of the present invention.

For example, the severity of Acne can be categorized into four degrees. First degree indicates mild Acne, e.g., pimples, mild papules and pus-filled pimples numbering less than 10. The treatment advice for first degree Acne includes treatment with topical acid or antibiotic ointment, optionally in combination with AHA chemical peeling treatment. Second degree indicates moderate Acne, e.g., 10-40 pimples and pus-filled pimples. The treatment advice for second degree Acne includes oral medication in addition to topical creams. Therefore, it is suggested to see a doctor. Third degree indicates moderate to severe Acne, e.g., 40-100 papules and pustules (more than 40 pimples), and a few nodular lesions (less than 3). It is highly suggested to see a doctor, and oral medication in addition to topical creams would be required. Fourth degree indicates severe Acne, e.g., multiple (more than 3) nodules and cysts combined with clustered lesions and papules and pustules. It would be necessary to see a doctor, and oral medication in addition to topical creams would be required.

In an embodiment, the instruction conducted by the smart medical application 10 to take new pictures of the patient in Step 23 can be given by using a large language model LLM (either at a remote end or at the edge) to talk to the patient or the photographer. Steps 22 and 23 are repetitively executed with the portable information device and the LLM until a required number of qualified face images is produced. For example, if a face image is determined to be out of focus in the quality check, the LLM may pop out a dialog window giving a “refocus on skin” instruction. In another example that a face image is determined to be shaky in the quality check, the LLM may pop out a dialog window giving a “turn on the anti-shake function” or “increase the shutter speed” instruction. In a further example that a face image is determined to be underexposed in the quality check, the LLM may pop out a dialog window giving a “pull up the ISO value” instruction. In this way, the required number of qualified face images can be efficiently collected.

For illustration only, a common smartphone 11 is used as an example to describe Step 24, i.e., the process of automatically combining the collected face images to create a planar panoramic face image of the patient, in detail hereinafter. First of all, when it is determined that a preset start key on the smartphone 11 is activated either by the patient himself or the person assisting in taking pictures, a visible and/or audible instruction is given through the smartphone 11 to instruct the user to take pictures of at least three orientations of the patient's face, e.g., right-side, front and left-side, thereby obtaining at least three face images including the right-side, front and left-side ones. Then the at least three face images are automatically analyzed and marked with a plurality of characteristic features, respectively. According to the plurality of characteristic features, the face images can be automatically combined to create the planar panoramic face image of the patient. The plurality of characteristic features at least includes a first set of features, which at least contains a right eyebrow tail feature, a right eye tail feature and a right mouth corner feature; a second set of features, which at least contains the right eyebrow tail feature, the right eye tail feature, the right mouth corner feature, a left eyebrow tail feature, a left eye tail feature and a left mouth corner feature; and a third set of features, which at least contains the left eyebrow tail feature, the left eye tail feature and the left mouth corner feature. Afterwards, the right-side face image and the front face image can be automatically combined into a first temporary image based on a virtual linking line of the right eyebrow tail feature, the right eye tail feature, and the right mouth corner feature, and the first temporary image automatically and extensively incorporates forehead and chin portions. Likewise, the left-side face image and the front face image can be automatically combined into a second temporary image based on a virtual linking line of the left eyebrow tail feature, the left eye tail feature, and the left mouth corner feature, and the second temporary image automatically and extensively incorporates forehead and chin portions. The first temporary image and the second temporary image are then combined into the planar panoramic face image.

In an embodiment, the first temporary image and the second temporary image can be automatically combined into the planar panoramic face image according to the virtual linking line of the right eyebrow tail feature, the right eye tail feature and the right mouth corner feature in the first temporary image and the virtual linking line of the left eyebrow tail feature, the left eye tail feature and the left mouth corner feature in the second temporary image.

In addition to the above mentioned virtual linking line of eyebrow tail features, eye tail features and mouth corner features, the technique to combine the three orientations of face images, e.g., right-side, front and left-side face images, may include alternative bases. For example, a virtual linking line of “temples-zygomatic arches-mouth corners-chin centerline”, a virtual linking line of “temporal hairline-eyebrow tail-eye tail-mouth corners-chin centerline”, a virtual linking line of “temporal hairline-eyebrow peak-pupil-mouth corners-chin centerline”; a virtual linking line of “forehead top hairline-eyebrow peak-pupil-mouth corners” (optionally extending downward to the chin), a virtual linking line of “temporal hairline-peak of the eyebrow-pupil-ala nasi-upper lip bulb-lip centerline-chin centerline”, and “eyebrow-temples of the eyes-ala nasi-mouth corner” (optionally extending upward to the forehead and downward to the chin). In addition to the technique of combining three orientations of face images, e.g., right-side, front and left-side face images, an alternative technique of combining two orientations of face images, e.g., right-side and left side face images can also be used. In this embodiment, a virtual linking line of “forehead centerline-centerline between eyebrows-nose bridge-nose tip-philtrum-lip centerline-chin centerline” can serve as the reference line for combination.

In the above embodiments, a plurality of multiple-orientation face images are generated and automatically combined into a planar panoramic face image. In an alternative embodiment, it is also feasible to directly generate a panoramic face image as long as the portable information device can support the required function. The panoramic face image, likewise, needs to be image-controlled with the image quality check model 100. If the panoramic face image fails in the quality check, it is required to take a new one with the same portable information device. Once the panoramic face image eventually passes the quality check, the panoramic face image is analyzed to automatically locate Acne lesions, count the number and classify the Acne lesions to obtain the Acne data file of the patient. Then the contents of the Acne data file are interpreted and analyzed by an artificial intelligence (AI) model, and accordingly, an inspection report relating to the facial skin condition is produced.

In an embodiment, a portable information device equipped with a Time-of-Flight (ToF) sensor can be used to take three-dimensional pictures from a plurality of orientations of face of the patient, thereby producing a first stereo model file of face image. The stereo model file of face image may contain a plurality of pixel points, each corresponding to a three-dimensional coordinate. The stereo model file of face image may also be checked with the image quality check model 100, and replaced with, for example, a newly taken stereo model file of face image when necessary. The process is repeated until a qualified one, e.g., second stereo model file of face image, is obtained. Subsequently, the three-dimensional coordinates of the pixel points included in the second stereo model file of face image are used for automatic pattern recognition, number counting and classification of Acne lesions to obtain the Acne data file of the patient. The recognition, counting and classifying processes may be performed by way of, for example, an image recognition model. Finally, an artificial intelligence model is used to interpret and analyze the contents of the acne data file to generate a report on the skin condition of the face. In an embodiment, a structured-light 3D scanner may also be used to accomplish the above 3D photography.

According to the present invention, automatic recognition of Acne lesions can be readily conducted by way of a portable information device such as a smartphone with an image quality check model, which may be built in the portable information device. The automatic recognition of Acne lesions can be performed on a planar panoramic face image. The recognized Acne lesions are automatically counted, classified and recorded. An Acne data file of the patient is thus established. Afterwards, the contents of the Acne data file can be interpreted and analyzed by an AI model. The results are recorded as an inspection report of facial skin condition of the patient. According to the inspection report, the severity of Acne lesions is evaluated. Based on the severity of Acne lesions, a medical advice about the need to see a doctor and/or a daily care advice about Acne care and skin maintenance will be given. In an embodiment of the present invention, the smart medical application 10 can be integrated into a portrait selfie application on the smartphone, and the smart medical application 10 integrated in the portrait selfie application can automatically perform the monitoring function in the background. In this way, when the user takes pictures on his daily lives with the portrait selfie application, or when the smartphone undergoes Face ID functions, face images can be simultaneously collected. The quality check of the face images can also be conducted in the background. According to the present invention, the AI model is made use of to automatically combine the required number of qualified face images into the planar panoramic face image, interpret and analyze the contents of the Acne data file correlating to the planar panoramic face image, and give proper advices and instructions. Furthermore, by correlating the monitoring method according to the present invention to the selfie application of the smartphone and recommended skin care products or services, it would be great help to the patient.

By using the method according to the present invention, all the above-described operations performed after taking the patient's pictures can be conducted automatically by way of the built-in functions of the portable information device and an image processing artificial intelligence (AI) model. The AI model may be built directly into the portable information device at the edge or may be a remote AI model. The built-in AI model may also be integrated with the smart medical application. The method according to the present invention is further advantageous in real-time inspection and advice. Furthermore, the Acne data file of the patient and associated reports may be de-identified and then uploaded to a data center 12 in the Cloud to be stored for subsequent tracing. As for the face images of the patient and the combined planar panoramic face image, it is preferred, but not limited, to be stored in only the portable information device or deleted in due course in order to protect the patient's privacy. It is also preferred that deep learning of the AI model used in the method according to the present invention is continuously conducted for better recognition and classification of Acne lesions in order to be applicable to other fields of skin treatment or care. Since the portable information device such as smartphone is basically as common as a daily necessity, the method according to the present invention is quite feasible as a medical assistant tool.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for monitoring a facial skin condition of a patient, comprising:
taking facial pictures of the patient at multiple orientations of the patient's face with a portable information device to produce a plurality of face images;
conducting a quality check of the plurality of face images with an image quality check model;
automatically giving an instruction to take a new facial picture for replacing a corresponding face image failing in the quality check;
repeating the steps of taking facial pictures and conducting the quality check until a required number of qualified face images are collected by the portable information device;
automatically combining the collected qualified face images into a planar panoramic face image;
automatically conducting a recognition process, a counting process and a classifying process of skin lesions of the patient based on the planar panoramic face image to generate a data file; and
interpreting and analyzing contents of the data file with an artificial intelligence model to produce an inspection report of the facial skin condition.

2. The method according to the present invention according to claim 1, wherein the quality check comprises a focus check, a hand-held shake check and/or an exposure value check.

3. The method according to the present invention according to claim 1, wherein the instruction to take the new facial picture is given via a large language model.

4. The method according to the present invention according to claim 1, wherein automatically combining the collected qualified face images into the planar panoramic face image comprises:
marking the collected qualified face images with a plurality of characteristic features, respectively; and
combining the collected qualified face images into the planar panoramic face image according to the plurality of characteristic features.

5. The method according to the present invention according to claim 1, wherein taking facial pictures of the patient at multiple orientations of the patient's face with the portable information device comprises:
issuing a visible and/or audible instruction to take pictures of the patient's face at multiple orientations to generate at least a first face image, a second face image and a third face image, respectively; and
automatically marking the first face image, the second face image and the third face image with a first set of characteristic features, a second set of characteristic features and a third set of characteristic features, respectively,
wherein the first face image, the second face image and the third face image are a right-side face image, a front face image and a left-side face image, respectively, and
wherein the first set of features at least contains a right eyebrow tail feature, a right eye tail feature and a right mouth corner feature, the second set of features at least contains the right eyebrow tail feature, the right eye tail feature, the right mouth corner feature, a left eyebrow tail feature, a left eye tail feature and a left mouth corner feature, and the third set of features at least contains the left eyebrow tail feature, the left eye tail feature and the left mouth corner feature.

6. The method according to the present invention according to claim 5, wherein automatically combining the collected qualified face images into the planar panoramic face image comprises:
automatically combining the right-side face image and the front face image into a first temporary image based on a virtual linking line of the right eyebrow tail feature, the right eye tail feature, and the right mouth corner feature, wherein the first temporary image automatically and extensively incorporates forehead and chin portions;
automatically combining the left-side face image and the front face image into a second temporary image based on a virtual linking line of the left eyebrow tail feature, the left eye tail feature, and the left mouth corner feature, wherein the second temporary image automatically and extensively incorporates forehead and chin portions; and
automatically combining the first temporary image and the second temporary image into the planar panoramic face image.

7. The method according to the present invention according to claim 6, wherein the first temporary image and the second temporary image into the planar panoramic face image are automatically combined based on the virtual linking line of the right eyebrow tail feature, the right eye tail feature and the right mouth corner feature in the first temporary image and the virtual linking line of the left eyebrow tail feature, the left eye tail feature and the left mouth corner feature in the second temporary image.

8. The method according to the present invention according to claim 1, wherein automatically conducting the recognition process, the counting process and the classifying process of skin lesions of the patient to generate the data file is conducted with an image recognition model.

9. The method according to the present invention according to claim 1, wherein the inspection report of the skin facial condition comprises a medical advice about the need to see a doctor, and a daily care advice about lesion care and skin maintenance.

10. A method for monitoring a facial skin condition of a patient, comprising:
creating a panoramic face image of the patient with a portable information device;
conducting a quality check of the panoramic face image with an image quality check model;
automatically giving an instruction to create a new panoramic face image for replacement if the quality check determines a failure result;
repeating the steps of creating the panoramic face image and conducting the quality check until the panoramic face image passes the quality check;
automatically conducting a recognition process, a counting process and a classifying process of skin lesions of the patient based on the panoramic face image passing the quality check to generate a data file; and
interpreting and analyzing contents of the data file with an artificial intelligence model to produce an inspection report of the facial skin condition.

11. A method for monitoring a facial skin condition of a patient, comprising:
taking three-dimensional pictures from a plurality of orientations of the patient's face with a portable information device, thereby producing a first stereo model file of face image, wherein the stereo model file of face image contains a plurality of pixel points, each corresponding to a three-dimensional coordinate;
conducting a quality check of the panoramic face image with an image quality check model;
repeating the steps of taking the three-dimensional pictures and conducting the quality check until a second stereo model file of face image, which passes the quality check, is obtained;
automatically conducting a recognition process, a counting process and a classifying process of skin lesions of the patient based on the second stereo model file of face image to generate a data file; and
interpreting and analyzing contents of the data file with an artificial intelligence model to produce an inspection report of the facial skin condition.
